(19) United States
(12) Patent Application Publication
(10) Pub. No.: US 2014/0114123 A1
(43) Pub. Date: Apr. 24, 2014

(54) Sex aid

(71) Applicant: Mart Mancillas, Dallas, TX (US)

(72) Inventor: Mart Mancillas, Dallas, TX (US)

(21) Appl. No.: 13/656,713

(22) Filed: Oct. 21, 2012

(51) Int. Cl. A61F 5/41

(52) U.S. Cl. 600/41

(57) ABSTRACT

A sex aid to aid in the sexual arousal of a user may include a center friction ring, a first strap to connect to the center friction ring and a pair of opposing support straps to extend around the buttocks of the user.

The center friction ring may be positioned over the genitals of a female user.

### FIELD OF THE INVENTION

The present invention relates to a sex aid and more particularly to a sex aid for men.

### BACKGROUND

Men sometimes find it difficult to be sexually aroused and may require additional stimulus in order to be sufficiently sexually aroused. Under these conditions, it may be difficult to provide sufficient satisfaction for the females.

It is an object of the present invention to provide a device to facilitate the sexual arousal of men.

### SUMMARY

A sex aid to aid in the sexual arousal of a user may include a center friction ring, a first strap to connect to the center friction ring and a pair of opposing support straps to extend around the buttocks of the user.

The center friction ring may be positioned over the genitals of a female user.

The first strap may include a first loop.

The first strap may include a second loop.

One of the support straps may include a first support loop.

One of the support straps may include a second support loop.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a perspective view of the sex aid device of the present invention;

FIG. 2 illustrates a perspective view of the sex aid device of the present invention;

FIG. 3 illustrates a front view of the sex aid device of the present invention;

FIG. 4 illustrates a front perspective view of the sex aid device and user of the present invention;

FIG. 5 illustrates a back perspective view of the sex aid device and user of the present invention;

FIG. 6 illustrates a side perspective view of the sex aid device and user of the present invention.

### DETAILED DESCRIPTION

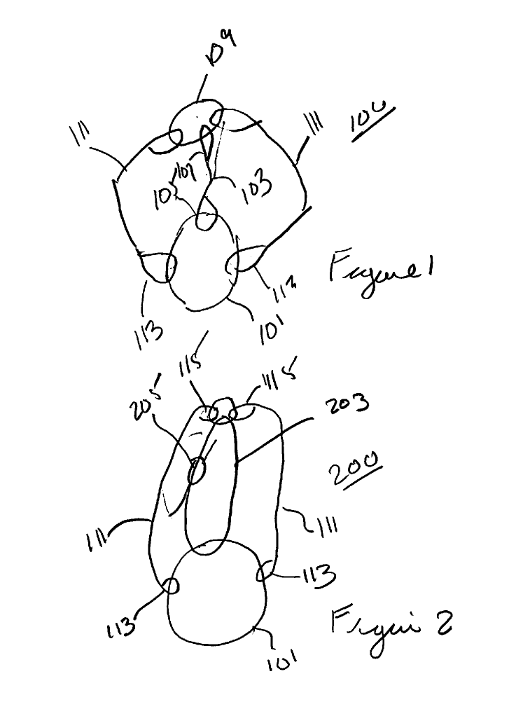

FIG. 1 illustrates the sex aid device 100 which may include a center friction ring 101 which may be rigid, semi-flexible or flexible in order to cooperate with the female sex organ (genitals) so that the male can insert his penis into the friction ring 101 which may be oval, round or other shape in order to encourage the penis to expand.

A first front strap 103 may include a first loop 105 at an end of the first front strap 105 to connect to the friction ring 101. The first loop 105 may travel around the friction ring 101, and the first front strap 103 may include a second loop 107 which may be formed on the opposite end of the first loop 105. The second loop 107 may connect to a support ring 109 so that the second loop 107 may travel around the support ring 109.

A pair of opposing support straps 111 may be positioned on each side of the first front strap 103 and may be substantially mirror straps. The support strap 111 may include a first support loop 113 at an end of the first support strap 111 to connect to the center ring 101 to allow the first support loop 113 to travel along the center ring 101 and may include a second support loop 115 at the opposing end of the first support strap 111 to connect to the support ring 109 which may be flexible or rigid.

The first front strap 103 and the support strap 111 may include a buckle to adjust the length of the respective front strap 103 and the support strap 111.

FIG. 2 illustrates the sex aid device 200 which may include a center friction ring 101 which may be rigid, semi-flexible or flexible in order to cooperate with the female sex organ (genitals) so that the male can insert his penis into the friction ring 101 in order to encourage the penis to expand.

A first front strap 203 which may be a loop connects to the friction ring 101 and may include a buckle 205. The front strap 203 may connect to a support ring 109 so that the front strap 203 may travel around the support ring 109.

A pair of opposing support straps 111 may be positioned on each side of the first front strap 203 and may be substantially mirror straps. The front strap and the support strap may formed from plastic, leather, steel wire with a protective coating which may be plastic or other material. The support strap 111 may include a first support loop 113 at an end of the first support strap 111 to connect to the center ring 101 to allow the first support loop 113 to travel along the center ring 101 and may include a second support loop 115 at the opposing end of the first support strap 111 to connect to the support ring 109 which may be flexible or rigid.

The front strap 303 and the support strap 111 may include a buckle to adjust the length of the respective front strap 103 and the support strap 111.

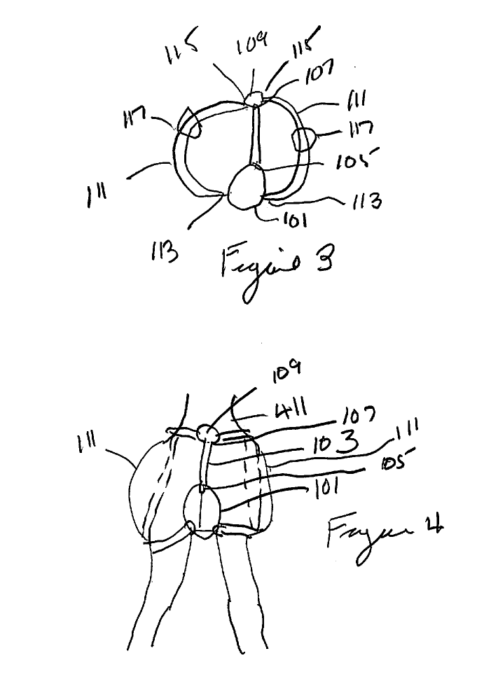

FIG. 3 illustrates the sex aid device 100 which may include a center friction ring 101 which may be rigid, semi-flexible or flexible in order to cooperate with the female sex organ (genitals) so that the male can insert his penis into the friction ring 101 in order to encourage the penis to expand.

A first front strap 103 may include a first loop 105 at an end of the first front strap 105 to connect to the friction ring 101. The first loop 105 may travel around the friction ring 101, and the first front strap 103 may include a second loop 107 which may be formed on the opposite end of the first loop 105. The second loop 107 may connect to a support ring 109 so that the second loop 107 may travel around the support ring 109.

A pair of opposing support straps 111 may be positioned on each side of the first front strap 103 and may be substantially mirror straps. The support strap 111 may include a first support loop 113 at an end of the first support strap 111 to connect to the center ring 101 to allow the first support loop 113 to travel along the center ring 101 and may include a second support loop 115 at the opposing end of the first support strap 111 to connect to the support ring 109 which may be flexible or rigid.

The first front strap 103 and the support strap 111 may include a buckle 117 to adjust the length of the respective front strap 103 and the support strap 111.

FIG. 4 illustrates the sex aid device 100 which may include a center friction ring 101 which may be rigid, semi-flexible or flexible in order to cooperate with the female sex organ (genitals) of the user 411 so that the male can insert his penis into the friction ring 101 in order to encourage the penis to expand.

A first front strap 103 may include a first loop 105 at an end of the first front strap 105 to connect to the friction ring 101. The first loop 105 may travel around the friction ring 101, and the first front strap 103 may include a second loop 107 which may be formed on the opposite end of the first loop 105. The second loop 107 may connect to a support ring 109 so that the second loop 107 may travel around the support ring 109. The support ring 109 may be positioned substantially at the waist of the user 411.

A pair of opposing support straps 111 may be positioned on each side of the first front strap 103 and may be substantially mirror straps. The support straps 111 may extend around the buttocks of the user 411. The support strap 111 may include a first support loop 113 at an end of the first support strap 111 to connect to the center ring 101 to allow the first support loop 113 to travel along the center ring 101 and may include a second support loop 115 at the opposing end of the first support strap 111 to connect to the support ring 109 which may be flexible or rigid.

The first front strap 103 and the support strap 111 may include a buckle to adjust the length of the respective front strap 103 and the support strap 111.

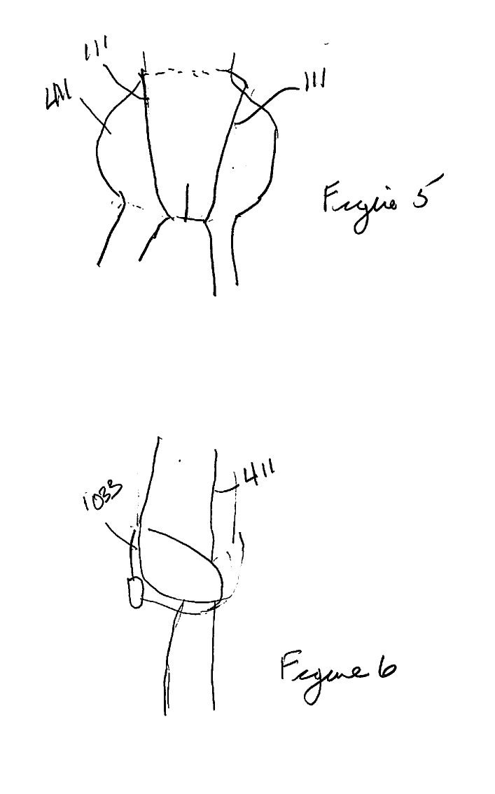

FIG. 5 illustrates a back view of the user 411 and illustrates the support straps 111.

FIG. 6 illustrates a side view of the user 411 and illustrates the front strap 103, the support strap 111, the center ring 101 and the support ring 109.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1. A sex aid to aid in the sexual arousal of a user, comprising:
a center friction ring;
a first strap to connect to the center friction ring;
a pair of opposing support straps to extend around the buttocks of the user;
wherein the center friction ring is positioned over the genitals of a female user.

2. A sex aid to aid in the sexual arousal of a user as in claim 1, wherein the first strap includes a first loop.

3. A sex aid to aid in the sexual arousal of a user as in claim 1, wherein the first strap includes a second loop.

4. A sex aid to aid in the sexual arousal of a user as in claim 1, where in one of the support straps includes a first support loop.

5. A sex aid to aid in the sexual arousal of a user as in claim 1, wherein one of the support straps includes a second support loop.
